(19) United States
(12) Patent Application Publication
(10) Pub. No.: US 2017/0151105 A1
(43) Pub. Date: Jun. 1, 2017

(54) A PACKAGING UNIT FOR HYGIENE ARTICLES AND A METHOD OF FORMING A PACKAGING UNIT

(71) Applicant: SCA HYGIENE PRODUCTS AB, Göteborg (SE)

(72) Inventor: Anders HOLMBERG, Göteborg (SE)

(73) Assignee: SCA HYGIENE PRODUCTS AB, Göteborg (SE)

(21) Appl. No.: 15/316,192

(22) PCT Filed: Jun. 13, 2014
(86) PCT No.: PCT/SE2014/050724

§ 371 Date: Dec. 5, 2016

(51) Int. Cl. A61F 13/551; B65B 11/48; B65B 61/06; B65B 51/02; B65D 75/20; B65D 65/14

(52) U.S. Cl. CPC A61F 13/5514; B65D 75/20; B65D 65/14; A61F 13/5515; B65B 61/06; B65B 51/02; B65B 11/48; B65D 2575/586

(57) ABSTRACT

A packaging unit for hygiene articles, the unit being formed from a sheet of material having a longitudinal direction and a transverse direction. The sheet has an inner surface and an outer surface. The inner surface includes an edge portion being provided with a resealable adhesive for closure of the packaging unit. Furthermore, the sheet has at least one transversely extending folding axis dividing said sheet into a first region and a second region. The sheet is provided with at least one sync mark for indication of a positioning of the sheet of material during manufacturing of the packaging unit, the sync mark being provided on the outer surface. A blank and a method for forming the packaging unit are also provided.

### TECHNICAL FIELD

The present disclosure relates to a packaging unit for hygiene articles. The present disclosure also relates to a blank of an elongated sheet material for providing a packaging unit, as well as to a method of forming a packaging unit for hygiene articles from a blank of an elongated sheet material.

### BACKGROUND

Disposable hygiene articles, such as sanitary napkins and panty liners, are normally packaged individually in, for example, an easy wrap or a single wrap. Individual packages facilitate hygienic carrying of single articles for future use, e.g. in a handbag. The edges of the individual packages can be sealed by means such as ultrasonic welding, heat welding and mechanical embossing with or without heat. Further, the packaging units are often used both as a means for packaging an unused article and for disposal of the used article.

It is desirable that used articles of this kind can be disposed of discretely and hygienically. This may be particularly important when the user lacks the possibility to dispose of the used article immediately after the used article has been replaced, e.g. when there is no waste bin available in the toilet area. In this case, the user may need to put the used article in e.g. a handbag or backpack, which requires that the package is adequately sealed in order to avoid staining and odour.

To solve the problem of providing a single wrap that may be both used for packaging of a new and unused hygiene article and for safe and hygienic disposal of used hygiene articles, the use of “post-it-like” adhesives has been shown to be useful as sealing means for the individual wrap packages.

WO2013/162430 A1 discloses such a packaging unit in the form a single wrap that can be used both for packaging of a new and unused hygiene article and for safe and hygienic disposing of used hygiene articles. The packaging unit is in the form of a sheet provided with adhesive in a certain chessboard pattern for closing and sealing the packaging unit.

Such a sheet may be fabricated by providing a sheet material on which, for example, adhesive is applied, and cutting the sheet into individual sheet of materials for forming the packaging unit.

Although said prior art packaging unit to some extent may alleviate the problems of providing a safe and hygienic packaging for unused as well as used hygiene articles, there is still a problem of providing adequate closure and sealing of the package by the use of adhesive, wherein it may be difficult to adhere the adhesive to a sheet surface during production of the packaging unit as well as during the use of the packaging unit.

Thus, there is still a need for further improvements of packaging units so as to provide a packaging unit that is easy and reliable to produce and handle, wherein the packaging unit may adequately enclose and seal an unused and a used hygiene article.

### SUMMARY

In view of known packaging units, it is an object of the present invention disclosure to provide an improved packaging unit arrangement for carrying new hygiene articles as well as used hygiene articles, which arrangement provides easy and reliable handling, while being easy to produce.

The object is wholly or partially achieved by a packaging unit according to appended claim 1, a blank according to claim 21 and a method according to appended claim 23. Embodiments are set forth in the appended dependent claims, in the following description and in the drawings.

Thus, there is provided a packaging unit for hygiene articles, the unit being formed from a sheet of material having a longitudinal direction (L) and a transverse direction (T). The sheet has an inner surface and an outer surface, wherein the inner surface comprises an edge zone that comprises an edge portion being provided with a resealable adhesive for closure of the packaging unit. The sheet has at least one transversely extending folding axis dividing the sheet into a first region and a second region. Furthermore, the sheet is provided with a sync mark for indication of a positioning of the sheet of material during manufacturing of the packaging unit. The sync mark is provided on the outer surface.

The packaging unit is in the form of a sheet that may be used as a single wrap that has resealable adhesive for closure of the packaging unit holding an unused a hygiene article therein or a used article therein.

As used herein, the term “inner surface” refers to the surface of the packaging unit facing the product positioned inside the packaging unit, and the term “outer surface” refers to the surface opposite to the inner surface, i.e. the surface facing the ambient.

By the term “edge zone” is meant the portion of the packaging unit adjacent to the edges of the packaging unit. The width of an edge zone may be varied. The edge zone may be divided into an inner edge portion and an outer edge portion. The term “inner edge portion” then refers to the portion of the edge zone positioned towards center of the packaging unit, such as towards the longitudinal center line of the packaging unit. The term “outer edge portion” refers to the portion of the edge zone positioned towards the edge of the packaging unit.

By a “resealable adhesive” as used herein is meant adhesive that provides that an object may be attached to a surface using such an adhesive and later on detached from that surface causing minor or no disruptions of the object and the surface. Furthermore, an object that has been peeled off may also be reattached to a surface again.

By the term “sync mark” is meant a position mark for synchronizing a positioning of the packaging unit and its parts during manufacturing thereof.

The sync mark provided on the outer surface may therefore be used as a mark for discerning the individual packing unit and regions thereof during production thereof. For example, it may be used as a positioning mark for indicating a cutting position of a blank sheet of material for providing a single packaging unit such as a wrap. Also, the sync mark may be used as a mark for examining whether, for example, the resealable adhesive is applied at a predetermined position on the packaging unit.

By providing the sync mark on the outer surface, an improved resealable adhesive connection to the inner surface is made possible and with no sync marks on the inner surface, the inner surface is also prepared for pre-treatments such as corona treatments for providing an improved resealable adhesive connection to the inner surface.

The sync mark may be a printed mark.

A printed mark is a mark printed to form a coloured mark, including a mark in black. The printed mark may be provided by a contact printing technique such as flexographic printing, rotogravure printing, screen printing and offset printing. The printed mark may also be provided by a non-contact printing technique such as ink jet printing, wax jet printing, bubble jet printing and laser jet printing.

At least a portion of the inner surface may have been pre-treated with a surface treatment for changing the surface energy.

The surface energy level corresponds to properties of the surface, in particular to the disruption of intermolecular bonds that occur when a surface is created or pre-treated. The pre-treatment modifies the surface properties, such as increasing the disruption of intermolecular bonds, and thereby increases the surface energy that in turn provides an improved adhesive connection.

The pre-treated portion may overlap with the edge portion comprising the resealable adhesive for closure of the packaging unit.

The surface treatment may be a plasma treatment. The plasma treatment may be a corona treatment. The corona treatment may be performed as is known in the field and described in US 2012/0232509 A1 wherein the treatment is applied at from 1 to 10, from 3 to 5 or 4 watts per square feet per minute. The corona-treated sheet of material should be subjected to application of adhesives and other agents shortly after the corona treatment of the sheet, preferably within 2 to 6 weeks from the corona treatment has been performed.

“Corona treatment” is a surface treatment technique that uses the so-called corona discharge plasma by the application of high voltage at low temperature to modify the properties of a surface. Materials such as plastics and nonwoven substrates may be corona treated so as to change the surface energy of such materials and to provide better bonding surfaces. The corona treatment increases the surface energy of the material and thereby improving the wettability and adhesion characteristics of, for example, adhesives to the materials such as polyolefin films and nonwoven substrates.

By providing the sync mark on the outer surface and pre-treating the inner surface with a corona treatment, there is provided an adequate adhesive connection to the corona-treated surface, as compared to providing the sync mark on the inner surface since any printing on the inner surface tends to interfere with the adhesive attachment, as the attachment to a corona-treated surface is stronger than to a printed surface, even if the printed surface portion has been corona treated.

A portion of the resealable adhesive for closure of the packaging unit may be in contact with a portion of the surface that has been pre-treated with surface treatment.

The complete inner surface portion to which the resealable adhesive is applied may be corona treated. The complete inner surface of the packaging unit sheet may also be pre-treated.

The sheet may be folded along the at least one folding axis with the first and second regions in an overlapping configuration, thereby providing a packaging unit of overlapping sheet layers.

The sheet of material may be of substantially rectangular shape and comprise longitudinal edges and transverse edges.

The sync mark may be provided at a longitudinal edge portion and/or at a transversal edge portion of the sheet of material.

The transverse direction (T) normally corresponds to a machine direction (MD) during manufacturing of the packaging unit. The longitudinal direction (L) then corresponds to the cross machine direction (CD). The cutting of the blank sheet of material may then be performed along the longitudinal direction, i.e. CD.

As mentioned above, the packaging unit comprises at least one first folding axis. The number of folding axes may vary depending on how the packaging unit is intended to be folded. It is preferred that the packaging unit comprises between one and three folding axes. The sheet may have two folding axes that divide the sheet into the first region, the second region and a third region.

The sheet may then be folded along the folding axes with the first, second and third regions in an overlapping configuration of three layers. The first region may form an outer layer of the folded packaging unit and the third region an inner layer. The sync mark may be provided on the first region, second region or third region.

The packaging unit may comprise two or more sync marks.

The sheet may a laminate material. By the term “laminate” is meant a material comprising at least two united separable plies of material that can be the same or different. In the context of the present disclosure, the laminate may for example be constituted of two separable plies of plastic film, a film and nonwoven, two plies of nonwoven or the like.

The sheet may be a single-ply material.

By the term “single ply” is meant a packaging unit comprising a single ply of a coherent material. Examples of a single ply packaging unit may be a plastic film, such as a polyethylene film, a nonwoven material, a metallic foil or the like. A single ply material may be a non-homogenous material, such as a plastic film material comprising integrated layers or a nonwoven material having varying fibre composition in different parts of the material. A single ply material as used herein does not comprise materials having separable layers.

The single-ply material may be of polyolefin that may comprise or be of polyethylene and/or polypropylene. The material of polyethylene and/or polypropylene may have a basis weight of 14 to 30 g/m2.

A chessboard pattern of resealable adhesive and non-adhesive portions may be formed along each edge zones for complementary attachments between resealable adhesive and non-adhesive portions in a folded packaging unit.

Examples of different chessboard patterns will be further described below. WO2013/162430 A1 discloses further examples of such patterns.

The sheet may be opaque and/or may comprise print and/or tactile indications, other than the sync marks, preferably provided on the outer surface. Thus, the sheet may, for example, be embossed.

The print may be instructions or indications that guide the user in the manipulation of the packaging unit and/or the hygiene article.

The resealable adhesive may be a pressure-sensitive hotmelt adhesive. Such an adhesive provides unlimited open time, meaning that the adhesive can bond to another substrate at any time. It is may provide “post-it-like” adhesive properties. Thus, the packaging unit may be reclosable.

Accordingly, the pressure-sensitive adhesive used with the packaging unit is one which has a very high self-adhesion but which can be readily separated or released from other materials, such as plastic materials or paper which has been treated with a release agent. A major advantage of the packaging unit according to the present disclosure is that it can be completely unfolded when a new hygiene article is about to be taken out. In contrast thereto, prior art packages having adhesively sealed edges with adhesive-coated edge portions in contact with each other have too high adhesive strength of the adhesively sealed edges, and any attempt to completely unfold the package generally leads to tearing and breakage of the packaging unit, making it unusable for discrete and hygienic disposal of the used article. As the adhesive-coated edge portions of the packaging unit of the present disclosure are not in contact with each other when the packaging unit is folded, the packaging unit can be readily opened and resealed, providing a tight disposal package. At the same time, the tensile strength of the adhesively sealed edges of the packaging unit using the resealable adhesive pattern of the present disclosure is sufficient to provide a tight package for both a new and a used article, and low enough to provide a readily-opened package.

The resealable adhesive used in the present disclosure may be a pressure-sensitive hotmelt adhesive, such as Lunatack® D656 BD 19 available from H. B. Fuller.

The length of the adhesive-covered edge portions in each region may be equal to or shorter than the length or width of each region. The width of the adhesive-covered edge portions may be varied depending on the adhesive strength desired. The wider the adhesive-covered edge zones, the stronger the sealing. The width of the adhesive-covered edge portions may be same or different in the different regions.

The sheet of material forming a packaging unit according to the present disclosure may comprise an odour-inhibiting or odour-neutralising substance. Such a substance may be applied in any suitable manner known to the person skilled in the art, e.g. as a coating, activatable microcapsules, impregnated patches, or the like.

It is conceivable that the sheet for forming a packaging unit according to the present disclosure may be stretchable or expandable, which may be advantageous if the hygiene article is greatly deformed during use, and may thus be difficult to wrap without deforming the packaging unit.

It should be noted that when the packaging unit according to the present disclosure is used for disposal, the user may choose to roll up the packaging unit instead of folding, regardless of the resealable adhesive pattern at the edge zone of the packaging unit.

The inner surface may comprise a center zone that comprises a portion provided with a release agent.

The term “central zone” refers to the portion of the packaging unit excluding the edge zone and overlapping with the center of the packaging unit.

The release agent is intended for releasable connection to an adhesive of a hygiene article in the packaging unit. The release agent may be a silicone polymer or polysiloxane.

As an alternative, a release liner may be attached to the central zone of the inner surface.

Such a release liner is also intended for releasable connection to a hygiene article. The release liner may be manufactured from any suitable material known to the person skilled in the art, such as paper, non-woven or plastic film material. Further, in order to provide a release liner that is easily detachable from the garment-affixing adhesive that may be present on a garment-facing surface of the hygiene article, the release liner may be silicone-coated on the surface of the release liner that is intended to be connected to the hygiene article. The hygiene article may be releasably affixed to the release liner either by means of a resealable adhesive or by any other suitable fastening means known to the person skilled in the art, e.g. hook-and-loop fastening means.

“Releasably affixed” means two surfaces being bonded to each other such that the bond may be readily broken without affecting the surfaces.

The packaging unit may comprise a hygiene article.

The hygiene article may be arranged on the inner surface, in the center zone thereof, wherein the hygiene article may be releasably attached to the release agent via an adhesive, or to the release liner.

There is also provided a blank for manufacturing at least two packaging units as described above. The blank is an elongated sheet of material having first and second surfaces, wherein the first surface comprises the inner surface of each packaging unit and the second surface comprises the outer surface of each packaging unit. The inner surface comprises a portion with resealable adhesive for closure of the packaging unit formed from the blank and the outer surface comprises one or more sync marks.

As mentioned above, the sync mark may be used for guiding a cutting tool to cut the blank into individual sheets of material and forming the packaging units.

A release agent in the form of, for example, a dispersion or layer may be applied on the first surface and inner surface, and a hygiene article may be arranged on the release agent.

At least a portion of the inner surface may have been pre-treated with a surface treatment for increasing the surface energy before any resealable adhesive has been applied on the first surface so as to improve adhesive connection to the pre-treated portion of the first surface.

The surface treatment may be a plasma treatment, such as a corona treatment.

There is also provided a method of forming a packaging unit for hygiene articles, the unit being formed from a blank of an elongated sheet of material. The method comprises the steps of:
providing the elongated sheet of material comprising at least two packaging unit sections, the sheet having first and second surfaces, the first surface comprising an inner surface of each packaging unit and the second surface comprising an outer surface of each packaging unit,
providing the outer surface with one or more sync marks,
applying a resealable adhesive on an edge zone portion of the inner surface of each packaging unit section;
detecting the sync mark and its position on the blank, and
cutting the elongated sheet into two or more single sheets or packaging units.

The position of cutting may be decided based on the detected position of the sync mark. Each portion with resealable adhesive may be arranged in an edge zone of a packaging unit section.

One or more sync mark detectors may be arranged to detect the sync mark on the outer surface during the manufacturing, wherein a detection of the sync mark is used for indicating a position of a blank sheet of material so as to determine, for example, a cutting position of the blank sheet of material for providing single packaging unit such as a wrap.

The method may further comprise a step of pre-treating at least a portion of the first surface with a surface treatment for increasing the surface energy.

The surface treatment may be a plasma treatment. The treatment may be a corona treatment.

The elongated sheet may comprise at least one folding axis extending in the machine direction (MD). The folding axis divides the elongated sheet into a first blank region and a second blank region. The method may further comprise folding the elongated sheet along the at least one folding axis with the first and second blank regions in an overlapping configuration, thereby providing a folded elongated sheet of overlapping sheet layers, wherein the step of cutting the elongated sheet occurs after the folding step.

The transversely extending folding axis may extend along the machine direction (MD) and the cutting may occur along the cross machine direction (CD).

Each single sheet formed after the step of cutting may comprise at least one transversely extending folding axis that divides each single sheet into a first region and a second region, wherein the method in a step following the method step of cutting the elongated sheet comprises folding the single sheet along the at least one folding axis with the first and second regions in an overlapping configuration, thereby providing a packaging unit of overlapping sheet layers.

The cutting may accordingly occur prior to the folding step. Nonetheless, the transversely extending folding axis may still extend along the machine direction (MD) and the cutting may occur along the cross machine direction (CD).

The formed packaging unit for hygiene articles may be a packaging unit as defined herein.

The method may, prior to the folding step, comprise:
applying a release agent on the first surface on a portion of each center zone of a packaging unit section,
applying a resealable adhesive on the portion with release agent, and
applying a hygiene article to the adhesive applied on the release agent.

Alternatively, the method may, prior to the folding step, comprise:
attaching a release liner on the first surface on a portion of each center zone of a packaging unit section and
connecting a hygiene article to the release liner by the use of fastening means.

Thus, the hygiene article may be releasably affixed to a surface of the release liner after the release liner has been permanently attached to the inner surface of the packaging unit. Alternatively, the hygiene article may be releasably affixed to the user-facing surface of the release liner before the release liner has been permanently attached to the inner surface of the wrapping sheet. The hygiene article may be releasably affixed to the release liner either by means of a resealable adhesive or by any other suitable fastening means known to the person skilled in the art, e.g. hook-and-loop fastening means.

The sync mark may aid in positioning of the applied agents and articles.

The present disclosure will now be described in more detail with reference to embodiments and drawings.

### BRIEF DESCRIPTION OF THE DRAWINGS

FIG. 1 shows a packaging unit according to an embodiment having one folding axis;

FIG. 2 shows the packaging unit depicted in FIG. 1 in a folded state;

FIG. 3 shows a packaging unit according to an embodiment having two folding axes;

FIG. 4 shows the packaging unit depicted in FIG. 3 in a folded state.

FIG. 5 schematically illustrates an arrangement for forming a packaging unit depicted in FIGS. 3 and 4.

### DETAILED DESCRIPTION OF EMBODIMENTS

The packaging unit of the present disclosure is in the form of a sheet that may be used as a single wrap that has resealable adhesive for closure of the packaging unit holding an unused hygiene article therein or a used article therein. The closure of the packaging unit may include folding the sheet and enclosing the hygiene article between folded sheet layers that are sealed by the resealable adhesive provided close to the edges of the sheets. Conceivable hygiene articles are sanitary napkins, panty liners and the like.

In the following, the packaging unit will be exemplified by a packaging unit comprising a rectangular sheet. Other forms such as circular sheet are also within the scope of the appended claims. Furthermore, the exemplified packaging unit comprises resealable adhesive for closure of the packaging unit, wherein a chessboard pattern of resealable adhesive and non-adhesive portions is formed along at least longitudinal edge zones (i.e. along the longitudinal direction (L)) for complementary attachment between resealable adhesive and non-adhesive portions in a folded packaging unit. According to the embodiment, other ways of applying one or more resealable adhesive portions for closure of the packaging unit may also be considered.

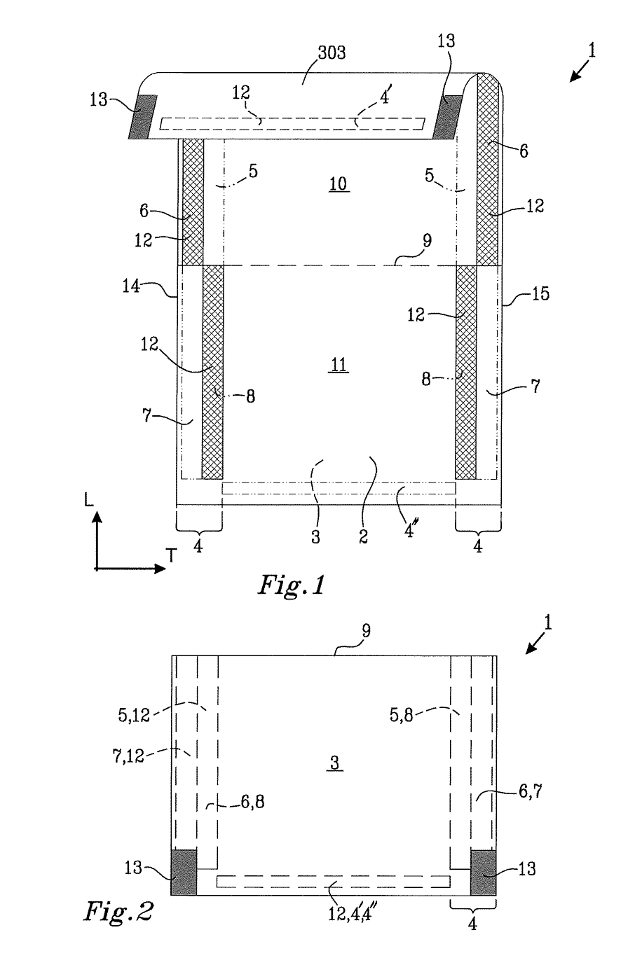

FIG. 1 depicts a packaging unit 1 for hygiene articles according to the present embodiment. The packaging unit is formed from a sheet of material having a longitudinal direction (L) and a transverse direction (T). The sheet has an inner surface 2 and an outer surface 3, the inner surface comprising an edge zone 4 comprising an inner edge portion 5, 8 and an outer edge portion 6, 7. The packaging unit 1 also comprises transverse edge portions 4′ and 4″. The sheet has a longitudinal center line (L1) and a folding axis 9, wherein the folding axis divides the sheet into a first region 10 and a second region 11. As shown in FIG. 1, the outer edge portion 6 of the edge zone 4 of the first region 10 is provided with resealable adhesive 12, while the inner edge portion 5 of the edge zone 4 of the first region 10 is adhesive-free. Further, the inner edge portion 8 of the edge zone 4 of the second region 11 is provided with resealable adhesive 12, while the outer edge portion 7 of the edge zone 4 of the second region 11 is adhesive-free. Also, the transverse edge portion 4′ of the first region 10 is provided with resealable adhesive 12, while the transverse edge portion 4″ of the second region 11 is adhesive-free. Thus, the resealable adhesive pattern in the first region 10 is complementary to the resealable adhesive pattern of the second region 11. This in turn means that, when the sheet is folded about the folding axis 9 as shown in FIG. 2, the edge portions 6 carrying resealable adhesive in the first region 10 are brought in contact with the adhesive-free edge portions 7 in the second region 11, the edge portions 8 carrying resealable adhesive 12 in the second region 11 are brought in contact with the adhesive-free edge portions 5 in the first region 10, and the transverse edge portion 4′ carrying resealable adhesive 12 in the first region 10 is brought in contact with the adhesive-free transverse edge portion 4″ in the second region 11. As can be seen from FIG. 2, the width of the sealing area corresponds to the sum of the widths of the resealable adhesive portions 6, 8. Such a sealing ensures a tight package for both a new and a disposed article.

Accordingly, the resealable adhesive (-s) is provided on edge portions such that when the sheet is folded about the folding axis 9, the resealable adhesive aids in the closure of the packaging unit 1 enclosing a hygiene article in a tight and hygienic way. FIG. 2 illustrates the folded sheet along the at least one folding axis with the first and second regions 10, 12 in an overlapping configuration, thereby providing a packaging unit 1 of overlapping sheet layers.

A set forth in FIGS. 1 and 2, the sheet is further provided with two sync marks 13 for indication of a positioning of the sheet of material during manufacturing of the packaging unit 1. Each sync mark 13 is provided on the outer surface 3 of the sheet at a corner portion of the first region 10. By providing the sync mark 13 on the outer surface 2 of the sheet, an improved adhesive connection to the inner surface 2 is made possible. This is because adhesive bonds less well to a printed surface. With no sync marks on the inner surface 2, the inner surface 2 can be prepared for pre-treatments, such as corona treatments, for providing an improved adhesive connection to the inner surface 4, as described herein.

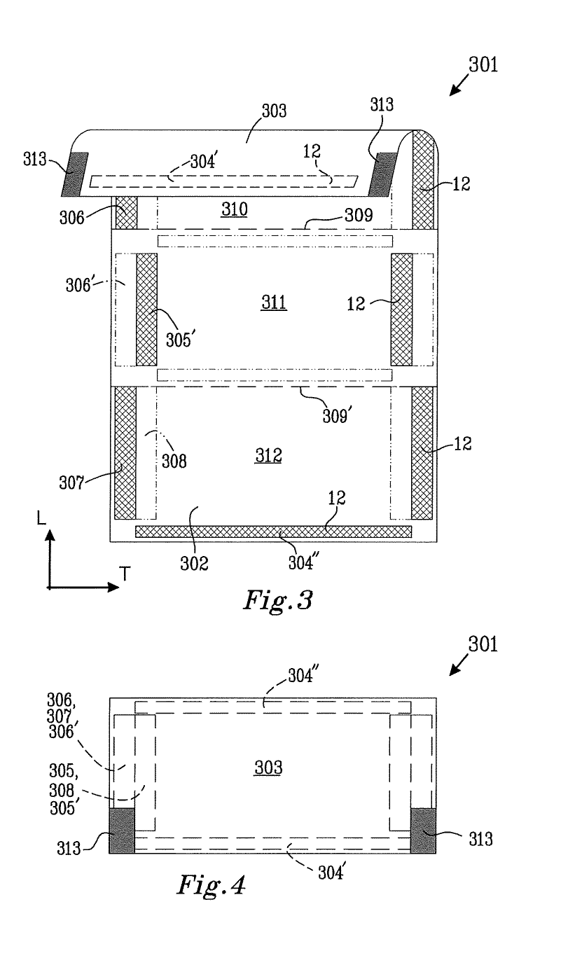

The most common packaging unit for individual packaging of absorbent articles is a rectangular sheet comprising two folding axes, longitudinal edges and transverse edges. Such an embodiment is illustrated in FIG. 3. The packaging unit 301 is a rectangular sheet comprising two folding axes 309, 309′ dividing the packaging unit into a first region 310, a second region 311 and a third region 312. Each of the regions comprises an inner edge portion 305, 305′, 308 and an outer edge portion 306, 306′, 307. As shown in FIG. 3, the outer edge portions 306, 307 of the first and third regions respectively are provided with resealable adhesive 12, while the inner edge portions 305, 308 of the first and third regions respectively are adhesive-free. The resealable adhesive pattern of the second region 311 is complementary to the resealable adhesive pattern of the first and third regions 310, 312, thus forming a chessboard pattern. In other words, the outer edge portion 306′ of the second region 311 is adhesive-free, and the inner edge portion 305′ of the second region 311 is provided with resealable adhesive. This in turn means that, when the sheet is e-folded about the folding axes 309, 309′ as shown in FIG. 4, the outer edge portions 306 of the first region 310 or the outer edge portions 307 of the third region 312 carrying resealable adhesive 12 are brought in contact with the adhesive-free outer edge portions 306′ in the second region 311, depending on which of the first and the third regions 310, 312 is brought in contact with the second region 311. Consequently, the inner edge portions 305′ carrying resealable adhesive 12 in the second region 311 are brought into contact with the adhesive-free inner edge portions 305 of the first region 310 or the adhesive-free inner edge portions 308 of the third region 312.

As can be seen from FIG. 4, the width of the sealing area corresponds to the sum of the widths of the resealable adhesive portions 306, 305′. It should be noted that the order in which the packaging unit is folded is irrelevant. For example, the packaging unit may be folded around the second folding axis 309′, bringing the third region 312 in contact with the second region 311, sealing the outer edge portions 307, 306′ and the inner edge portions 308, 305′. The packaging unit is subsequently folded around the first folding axis 309′, bringing the first region 310 in contact with the outer surface of the third region 312, thus sealing the packaging unit (FIG. 4). The folding order may also be reversed. This is a great advantage, since when the packaging unit of the present embodiment is used for disposal; the user does not have to fold the packaging unit in any particular order to be able to obtain a tightly sealed package. Thus, the packaging unit will provide a tight and hygienic package regardless of the folding order.

In order to obtain a tight package, the transverse edge portions 304′ and 304″ of the packaging unit 301 are provided with resealable adhesive 12. When both transverse edges 304′ and 304″ are provided with resealable adhesive, the folding order is irrelevant, as described above. It is also conceivable to provide only one of the transverse edges with resealable adhesive. In this case, the folding should be initiated around the folding axis being positioned closest to the adhesive-free transverse edge, such that the region comprising the adhesive-covered transverse edge portion forms a lid and the resealable adhesive positioned at the transverse edge portion seals the packaging unit.

The sheet as set forth in FIGS. 3 and 4 is also provided with two sync marks 313 for indication of a positioning of the sheet of material during manufacturing of the packaging unit. Each sync mark 313 is provided on the outer surface 303 of the sheet at a corner portion of the first region 310. As for the single-axis sheet, the provision of the sync mark 313, such as a printed sync mark, on the outer surface 303 means that an improved adhesive connection to the inner surface 302 is made possible as further described herein.

The use of marks for indicating, for example, cutting positions of a product is known from, for example, WO 03030796 A1.

It should be noted that when the packaging unit according to the present embodiment is used for disposal, the user may choose to roll up the packaging unit 1, 301 with the soiled article positioned on it rather than folding it.

The packaging unit is intended to include a hygiene article. The hygiene article may be arranged on the inner surface, in the center zone thereof, wherein the hygiene article may be attached to the release agent via an adhesive or to a release liner(not shown), as now will be further elaborated on in the described example of a method of forming the packaging unit.

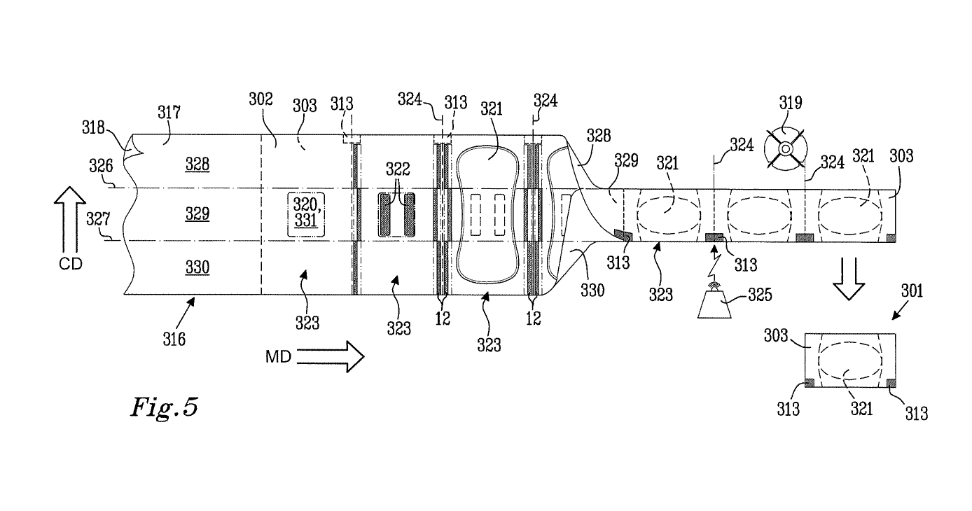

The packaging units may be formed from a blank. FIG. 5 schematically illustrates an arrangement of using a blank 316 for forming a packaging unit 301 with two folding axes 309, 309′ in accordance with the packaging unit 301 shown in FIGS. 3 and 4. The blank 316 is an elongated sheet of material having first and second surfaces 317, 318, wherein the first surface comprises the inner surface 302 of each packaging unit 301 and the second surface 318 comprises the outer surface 303 of each packaging unit 301. The inner surface is provided with resealable adhesive 12 for closure of the packaging unit 301 formed from the blank 316, and the outer surface 303 is provided with one or more sync marks 313. As mentioned above, such a sync mark 313 may be used for guiding a cutting tool 319 to cut the blank 316 into individual sheets of material and forming the packaging units 301.

A release agent 320 or a release liner may be applied on the first surface 317 and inner surface 302, and a hygiene article 321 may be arranged on the release agent 320 via an adhesive 322 or on the release liner via fastening means.

At least a portion of the first surface 317 and the inner surface 302 may be pre-treated with a surface treatment for increasing the surface energy before any adhesive has been applied on the first surface so as to improve adhesive connection to the pre-treated portion of the first surface 317 and the inner surface 302. The surface treatment may be a plasma treatment, such as a corona treatment as is described in more detail herein above.

The method used in the arrangement as illustrated in FIG. 5 then comprises the steps of:
providing a blank 316 of an elongated sheet of material comprising at least two packaging unit sections 323, the sheet having first and second surfaces 317, 318, the first surface 317 comprising an inner surface 302 of each packaging unit 301 and the second surface 318 comprising an outer surface 303 of each packaging unit 301,
providing the outer surface 303 with one or more sync marks 313, e.g. one or more printed marks;
applying a resealable adhesive 12 on an edge zone portion of the inner surface 302 of each packaging unit section 323;
detecting the sync mark 313 and its position on the blank 316, and
cutting the elongated sheet along a cutting line 324 into two or more packaging units 301.

The position of cutting and thus the cutting line may be determined based on the detected position of the sync mark (-s) 313.

In FIG. 5, each sync mark 313 is provided on the outer surface 303 of the sheet at a corner portion of the first region 310. Each sync mark 313 is applied on the blank as a combined sync mark overlapping the cutting line 324, and thus the combined sync mark forms two sync marks 313 after the blank 326 has been cutted, wherein a sync mark 313 is formed on each packaging unit.

Each portion with resealable adhesive 12 may be arranged in a zone close to and along the cutting line 324 of a packaging unit section 323, wherein the resealable adhesive 12 in some portions may be applied as one adhesive string (not shown) along and overlapping the cutting line 323 so that the applied string forms a portion with resealable adhesive 12 on each one of two packaging units 301 after cutting has taken place (not shown). The resealable adhesive 12 may also be separately applied to each packaging unit section 323 at a distance from the cutting line 324.

One or more sync mark detectors may be arranged to detect sync marks on the outer surfaces 303 during the manufacturing, wherein detection of the sync marks is used for indicating a position of a blank 316 sheet of material so as to determine, for example, a cutting position of the blank sheet of material for providing single packaging unit such as a wrap. FIG. 5 illustrates one detector 325.

As mentioned herein above, at least a portion of the first surface 317 may be pre-treated with a surface treatment for increasing the surface energy The surface treatment may be a plasma treatment. The treatment may be a corona treatment.

The elongated sheet may comprise at least one folding axis 326, 327 extending in the machine direction (MD). In FIG. 5, the elongated sheet has two folding axes 326, 327 dividing the elongated sheet into a first blank region 328, a second blank region 329 and a third blank region 330. The method illustrated in FIG. 5 further comprises folding the elongated sheet along the folding axes 326, 327 with the blank regions in an overlapping configuration, thereby providing a folded elongated sheet of overlapping sheet layers, wherein the step of cutting the elongated sheet occurs after the folding step. The transversely extending folding axes 326, 327 extend along the machine direction (MD) and the cutting occurs along the cross machine direction (CD). The folding is provided by any folding tool as is well known in the art (not shown).

The cutting may also occur prior to the folding step (not shown). Nonetheless, the transversely extending folding axis may still extend along the machine direction (MD) and the cutting may occur along the cross machine direction (CD).

Prior to the folding step, the method as illustrated in FIG. 5 further comprises:
applying a release agent 320 on the first surface 317 on a portion of each center zone 331 of a packaging unit section 323 (and corresponding packaging unit 301),
applying an adhesive 322 on the portion with release agent 320, and
applying a hygiene article 321 to the adhesive 322 applied on the release agent 320.

The sync mark (-s) 313 may also aid in positioning of the applied agents and articles.

As an alternative to use a release agent 320 and on the first surface 317 as illustrated in FIG. 5, the method may, prior to the folding step, further comprise:
attaching a release liner on the first surface 317 on a portion of each center zone 331 of a packaging unit section 323 and
connecting a hygiene article 321 to the release liner by the use of fastening means.

As the skilled person will appreciate, it is intended that the detailed description be regarded as illustrative and that many embodiments and alternatives are possible within the scope of the present disclosure as defined by the appended claims. For example, the packaging unit may adopt other shapes than the rectangular ones shown in the drawings. Furthermore, the present disclosure has been exemplified with packaging units comprising resealable adhesive for closure of the packaging unit, wherein a chessboard pattern of resealable adhesive and non-adhesive portions is formed along at least longitudinal edge zones (i.e. along the longitudinal direction (L)) for complementary attachments between resealable adhesive and non-adhesive portions in a folded packaging unit. Other ways of applying one or more resealable adhesive portions for closure of the packaging unit may also be considered.

1. A packaging unit for hygiene articles, the packaging unit being formed from a sheet of material having a longitudinal direction and a transverse direction, said sheet having an inner surface and an outer surface, said inner surface comprising an edge zone that comprises an edge portion being provided with a resealable adhesive for closure of the packaging unit, said sheet having at least one transversely extending folding axis, said at least one folding axis dividing said sheet into a first region and a second region, wherein said sheet is provided with at least one sync mark for indication of a positioning of the sheet during manufacturing of the packaging unit, said sync mark being provided on the outer surface.

2. The packaging unit unit according to claim 1, wherein the sync mark is a printed mark.

3. The packaging unit unit according to claim 1, wherein at least a portion of the inner surface is pre-treated with a surface treatment for changing a surface energy.

4. The packaging unit unit according to claim 3, wherein the surface treatment is a plasma treatment.

5. The packaging unit unit according to claim 3 4, wherein a portion of said resealable adhesive is in contact with a portion of the inner surface that has been pre-treated with surface treatment.

6. The packaging unit to claim 1, wherein said sheet is folded along said at least one folding axis with said first region and said second region in an overlapping configuration, thereby providing a packaging unit of overlapping sheet layers.

7. The packaging unit unit according to claim 1, wherein said sheet is of substantially rectangular shape and comprises longitudinal edges and transverse edges.

8. The packaging unit unit according to claim 7, wherein the sync mark is provided at at least one of a longitudinal edge portion and a transversal edge portion.

9. The packaging unit according to claim 7, wherein said sheet has two folding axes, dividing said sheet into said first region, said second region and a third region.

10. The packaging unit according to claim 9, wherein said sheet is folded along said two folding axes with said first region, said second region and said third region in an overlapping configuration of three layers.

11. The packaging unit according to claim 9, wherein the first region forms an outer layer of the folded packaging unit and the third region forms an inner layer, and wherein the at least one sync mark is provided on one of the first region, the second region and the third region.

12. The packaging unit unit unit according to claim 1, comprising two or more sync marks.

13. The packaging unit according to claim 1, wherein the sheet is a single-ply material.

14. The packaging unit according to claim 13, wherein the single-ply material is of polyolefin.

15. The packaging unit according to claim 1, wherein a chessboard pattern of the resealable adhesive and non-adhesive portions is formed along the edge zone for complementary attachment between the resealable adhesive and the non-adhesive portions in a folded packaging unit.

16. The packaging unit according to claim 1, wherein said sheet is at least one of:
opaque, and/or
comprises print, and
comprises tactile indications other than the sync mark.

17. The packaging unit according to claim 1, wherein said resealable adhesive is a pressure-sensitive hot melt adhesive.

18. The packaging unit according to claim 1, wherein said packaging unit is reclosable.

19. The packaging unit according to claim 1, wherein the inner surface comprises a center zone that comprises a portion provided with one of a release agent and a release liner.

20. The packaging unit according to claim 1, wherein the packaging unit comprises a hygiene article.

21. A blank for manufacturing at least two packaging units, each packaging unit being defined as claimed in claim 1, wherein the blank is an elongated sheet of material having a first surface and a second surface, the first surface comprising the inner surface of each packaging unit and the second surface comprising the outer surface of each packaging unit, wherein said inner surface comprises a portion with the resealable adhesive for closure of tho each packaging unit formed from the blank and wherein said outer surface comprises one or more sync marks.

22. The blank according to claim 21, wherein at least a portion of the inner surface is pre-treated with a surface treatment for increasing a surface energy.

23. A method of forming a packaging unit for hygiene articles, the packaging unit being formed from a blank of an elongated sheet of material, the method comprising the steps of:
providing said elongated sheet of material comprising at least two packaging unit sections, said elongated sheet of material having having a first surface and a second surface, the first surface comprising an inner surface of each packaging unit and the second surface comprising an outer surface of each packaging unit,
providing said outer surface with one or more sync marks,
applying a resealable adhesive on an edge zone portion of the inner surface of each packaging unit section;
detecting the one or more sync marks and their position on the blank, and
cutting said elongated sheet of material into two or more single sheets or packaging units.

24. The method according to claim 23, further comprising a step of pre-treating at least a portion of the first surface with a surface treatment for increasing a surface energy before said resealable adhesive is applied on the first surface.

25. The method according to claim 24, wherein the surface treatment is a plasma treatment.

26. The method according to claim 23, wherein the elongated sheet of material comprises at least one folding axis extending in a machine direction, said at least one folding axis dividing said elongated sheet of material into a first blank region and a second blank region, wherein the method further comprises folding said elongated sheet of material along said at least one folding axis with said first blank region and said second blank region in an overlapping configuration, thereby providing a folded elongated sheet of overlapping sheet layers, wherein said step of cutting the elongated sheet occurs after the folding step.

27. The method according to claim 23, wherein each single sheet formed after said step of cutting comprises at least one transversely extending folding axis, said at least one transversely extending folding axis dividing each single sheet into a first region and a second region, wherein the method further comprises, following the step of cutting the elongated sheet of material, folding said single sheet along said at least one folding axis with said first region and said second region in an overlapping configuration, thereby providing a packaging unit of overlapping sheet layers.

28. The method according to claim 26, wherein the formed packaging unit is for hygiene articles wherein the elongated sheet of material has at least one transversely extending folding axis, said at least one folding axis dividing said elongated sheet of material into a first region and a second region, wherein said one or more sync mark indicate a positioning of the elongated sheet of material during manufacturing of the packaging unit.

29. The method according to claim 26, wherein the method, prior to the folding step, further comprises
applying a release agent on said first surface on a portion of each center zone of a packaging unit section,
applying an adhesive on the portion with release agent, and
applying a hygiene article to said adhesive applied on the release agent.

30. The method according to claim 26, wherein the method, prior to the folding step, further comprises:
attaching a release liner on the first surface on a portion of each center zone of a packaging unit section, and
connecting a hygiene article to the release liner by the use of fastening means.
